(19) United States
(12) Patent Application Publication
(10) Pub. No.: US 2007/0082329 A1
(43) Pub. Date: Apr. 12, 2007

(54) Infertility assessment and treatment system

(75) Inventors: Russel H. Williams, Houston, TX (US); Rakesh Mangal, Houston, TX (US); David Nienberg, Houston, TX (US); David Stump, Houston, TX (US); Daniel Sharber, Houston, TX (US)

Correspondence Address: OSHA LIANG L.L.P., 1221 MCKINNEY STREET, SUITE 2800, HOUSTON, TX, 77010, US

(73) Assignee: Pathways to Pregnancy LP, Houston, TX (US)

(21) Appl. No.: 11/544,516

(22) Filed: Oct. 6, 2006

Related U.S. Application Data
(60) Provisional application No. 60/724,478, filed on Oct. 7, 2005.

(51) Int. Cl. G06Q 50/00; C12Q 1/00

(52) U.S. Cl. 435/4; 705/2

(57) ABSTRACT

A method for assessing fertility for a couple that includes obtaining semen analysis results from a male member of the couple, obtaining fertility survey responses from at least a female member of the couple, determining an initial location in a diagnostic matrix using the semen analysis results and the fertility survey responses, and obtaining a plurality of treatment options corresponding to the initial location in the diagnostic matrix.

### CROSS REFERENCE TO RELATED APPLICATIONS

This application claims benefit of U.S. Provisional Application Ser. No. 60/724,478 filed on Oct. 7, 2005, entitled “Infertility Assessment and Treatment” in the names of Russel H. Williams, Rakesh Mangal, David Nienberg, David Stump, and Daniel Sharber.

### BACKGROUND

Infertility may be due to a variety of causes. For example, as couples age (or more particularly the female members' age) the probability of conceiving decreases. Further, medical conditions such as genetic and/or abnormalities may also result in a decrease in a couple's ability to conceive. Environmental and lifestyle factors may also play a part in the couple's infertility. Examples of environmental and lifestyle factors include stress, exposure to toxins, etc.

The traditional manner to address a couple's inability to conceive is to select one member of the couple, typically the female member, and focus all medical attention on this member in an effort to increase that member's ability to conceive. Usually when the above approach fails, the couple usually chooses to pursue more expensive and complicated procedures, such as in vitro fertilization (IVF) or Artificial Insemination (AI).

### SUMMARY

In general, in one aspect, the invention relates to a method for assessing fertility for a couple comprising obtaining semen analysis results from a male member of the couple, obtaining fertility survey responses from at least a female member of the couple, determining an initial location in a diagnostic matrix using the semen analysis results and the fertility survey responses, and obtaining a plurality of treatment options corresponding to the initial location in the diagnostic matrix.

In general, in one aspect, the invention relates to a system for assessing fertility for a couple comprising a fertility survey component configured to obtain responses of a fertility survey from at least a female member of the couple, a semen analysis component configured to obtain results of semen analysis from a male member of the couple, and a diagnostic matrix component comprising a diagnostic matrix and configured to determine an initial location in a diagnostic matrix using the semen analysis results and the fertility survey responses, and obtain a plurality of treatment options corresponding to the initial location in the diagnostic matrix.

In general, in one aspect, the invention relates to a computer readable medium comprising instructions to obtain semen analysis results from a male member of the couple, obtain fertility survey responses from at least a female member of the couple, determine an initial location in a diagnostic matrix using the semen analysis results and the fertility survey responses, obtain a plurality of treatment options corresponding to the initial location in the diagnostic matrix, rank the plurality of treatment options, track progress of the couple as the couple pursues at least one of the plurality of treatment options, and move the couple from the initial location in the matrix to a new location in the matrix based on the progress of the couple.

Other aspects of the invention will be apparent from the following description and the appended claims.

### BRIEF DESCRIPTION OF DRAWINGS

FIGS. 1 and 2 show systems in accordance with one embodiment of the invention.

FIGS. 3A-3B show diagnostic matrices in accordance with one embodiment of the invention.

FIGS. 4A-4F show flowcharts in accordance with one embodiment of the invention.

FIG. 5 shows a flowchart in accordance with one embodiment of the invention.

FIG. 6 shows a computer system in accordance with one embodiment of the invention.

### DETAILED DESCRIPTION

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid obscuring the invention.

In general, embodiments of the invention relate to a method and system of assessing a couple's infertility and determining one or more treatment options to treat the infertility. More specifically, embodiments of the invention use the results of semen analysis and a fertility survey to determine a couple's initial location in a diagnostic matrix. Once the couple's initial location is determined, one or more treatment options associated with the initial location are presented to the couple. In some embodiments the treatment options are ranked based on the various couple-specific factors prior to being presented to the couple. The system also includes functionality to track the couples' progress (e.g., whether the couple has conceived, whether the female's ovulation has normalized, whether the male has an increased sperm count, etc.) as they follow one or more of the treatment options and adjust the couple's location in the diagnostic matrix based on how the couple is progressing.

Embodiments of the invention provide a method and apparatus for a couple to maximize their overall fertility, thereby increasing their chances of conceiving. In addition, embodiments of the invention provide a method and apparatus for effectively addressing fertility with respect to the couple as a whole.

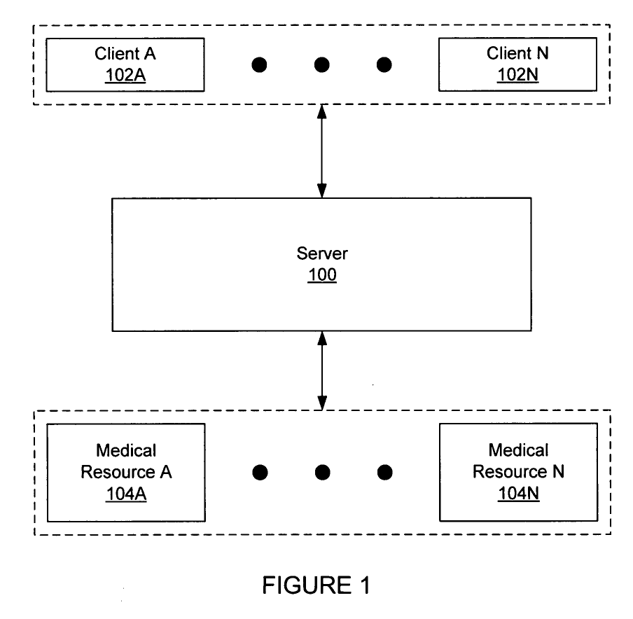

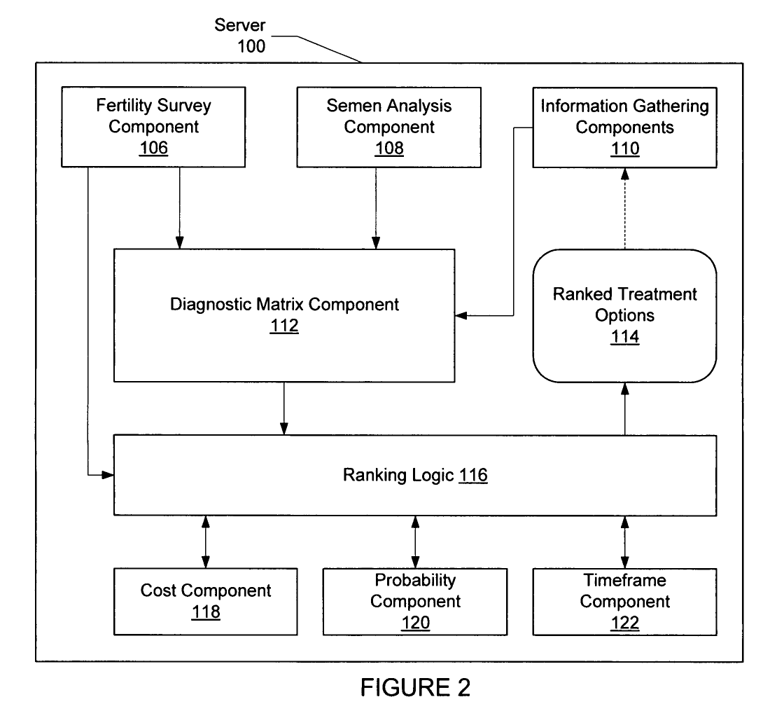

FIG. 1 shows a system in accordance with one embodiment of the invention. The system shown in FIG. 1 includes a server (100) operatively connected to one or more clients (102A, 102N). Further, the server (100) is also operatively connected to one or more medical resources (104A, 104N). In one embodiment of the invention, the server (100) corresponds to one or more computer systems that include functionality to obtain information from and present information to the clients (102A, 102N). In addition, the server (100) is configured to enable clients (102A, 102N) to interact with the medical resources (104A, 104N). The server (100) is discussed in FIG. 2 below.

In one embodiment of the invention, each client (102A, 102N) corresponds to a system configured to enable a user (i.e., one or both members of a couple) to interact with the server (100) (i.e., provide information to and receive information from the server (100)). For example, the client (102A, 102N) may correspond to a desktop computer in a user's home. Alternatively, the client (102A, 102N) may correspond to a computer in a public location, such as a hospital, clinic or community health center, that is accessible to a large number of users. The aforementioned examples are not intended to limit the scope of the invention.

In one embodiment of the invention, each medical resource (104A, 104N) corresponds to a system configured to perform at least one of the following functions: (i) provide medical information (e.g., a medical database, a portal to fertility websites, etc.) to a user; (ii) provide an interface (e.g., a virtual office, a chat room, bulletin board, a web log, etc.) between the user (e.g., the female member, the male member, and/or the couple) and a medical professional (e.g., gynecologist, urologist, an in vitro fertilization (IVF) specialist, etc.); (iii) allow the female member's doctor/specialist to communicate with the male member's doctor/specialist; (iv) provide a central repository for medical records associated with the couple that are accessible to the couple as well as the doctor(s) (including specialists) working to treat the couple; and (v) provide an interface (e.g., web portal) to enable a user to book medical procedures (e.g., an initial gynecologist examination, etc.) and/or tests (e.g., semen analysis test). Those skilled in the art will appreciate that the medical resources (104A, 104N) are not limited to the aforementioned functions.

Though not shown in FIG. 1, the server (100) may also include functionality to accept payments for various services offered by one or more of the medical resources (104A, 104N). In addition, the server (100) may include functionality to allow each user (e.g., a couple) to track the amount of money spent on services offered by one or more of the medical resources (104A, 104N).

FIG. 2 shows a system in accordance with one embodiment of the invention. As shown in FIG. 2, the server (100) includes a fertility survey component (106), a semen analysis component (108), information gathering components (110), a diagnostic matrix component (112), ranking logic (116), a cost component (118), a probability component (120), and a timeframe component (122). Each of the aforementioned components is described below.

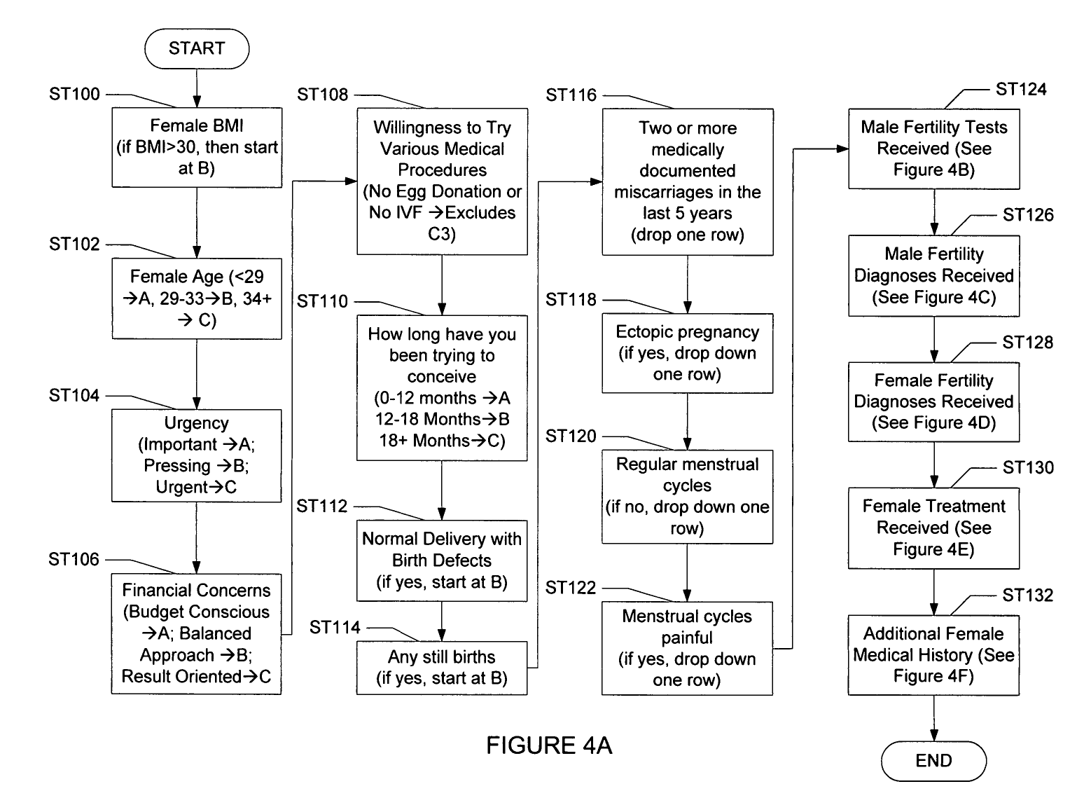

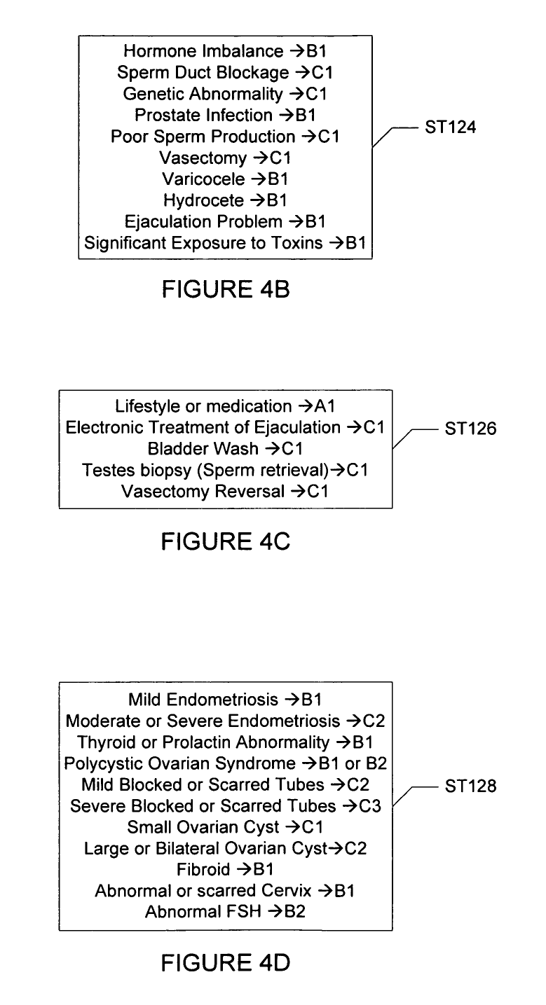

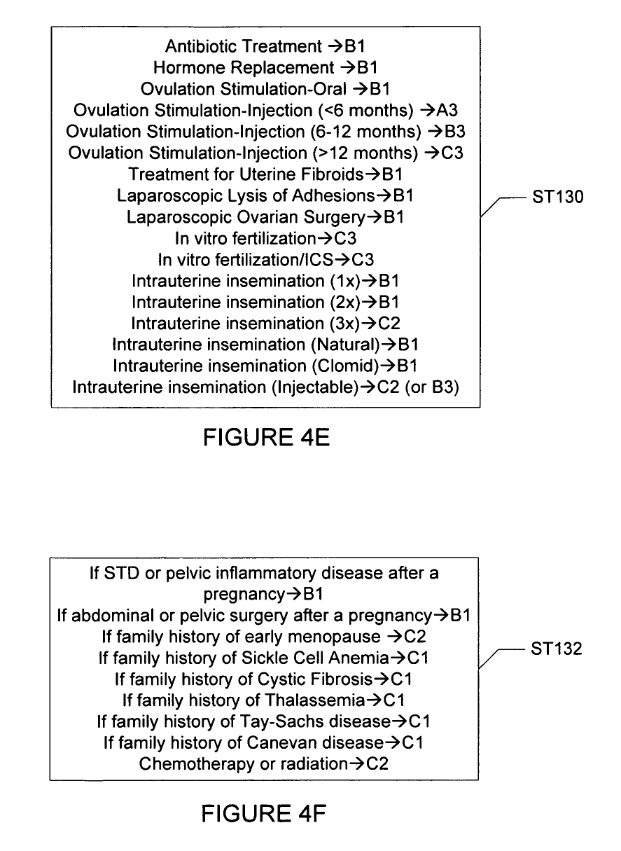

In one embodiment of the invention, the fertility survey component (106) includes a fertility survey and functionality to administer the fertility survey. In one embodiment of the invention, the fertility survey corresponds to one or more questions designed to obtain specific information about the couple (i.e., the male member and female member) to assess their infertility and determine a treatment option(s) in order to diagnosis one or more sources of infertility associated with the couple. An embodiment of the fertility survey is shown in FIG. 4A-4F and described below.

In one embodiment of the invention, the fertility survey component (106) administers the fertility survey by sending the entire survey to the user (via the client), allowing the user to complete the fertility survey (typically using the client), and receiving the completed fertility survey from the user (via the client). Upon receiving the user's responses to the fertility survey, the fertility survey component (106), if necessary, converts the user's responses into a format that is understood by the diagnostic matrix component (112). The responses (or in some cases the converted responses) are subsequently forwarded to the diagnostic matrix component (112) for analysis.

Alternatively, the fertility survey component (106) may conduct the fertility survey as follows. The user (via the client) is presented with a web-page that includes one or more questions. Further, for each question there is a means (e.g., a check box(es), a radio button(es), a text box(es), etc.) for the user to answer the question. Once the user has answered the questions on the web-page, the user is instructed to click a button to obtain the next set of questions. Upon clicking the button, the user's responses to the questions are sent to the fertility survey component (106). Upon receiving the user's responses, the fertility survey component (106) records the user's responses and then proceeds to send a web-page with the next set of questions to the user (via the client). The above process is repeated until the user has completed the fertility survey. As discussed above, the user's responses are converted, if necessary, prior to being sent to the diagnostic matrix component (112) for analysis.

Those skilled in the art will appreciate that the user may correspond to the female member of the couple or the male member of the couple, depending on the questions asked in the survey (i.e., some questions are directed to the female member of the couple and thus are answered by her, similarly, some questions are directed to the male member of the couple, and thus, are answered by him).

Continuing with the discussion of FIG. 2, in one embodiment of the invention the semen analysis component is configured to obtain at least the following information regarding the results of the semen analysis: total sperm count, percentage of motile sperm, percentage of sperm with normal morphology (i.e., normal shape), whether white blood cells (i.e., leucocytes) were present in the semen sample analyzed during the semen analysis, whether red blood cells were present in the semen sample analyzed during the semen analysis, whether bacteria was present in the semen sample analyzed during the semen analysis, whether a low volume of semen was provided in the semen sample analyzed during the semen analysis, whether agglutination is present, and sperm DNA results.

In one embodiment of the invention, the semen analysis component (108) obtains the aforementioned semen analysis results by providing the user with a web-page that includes a series of input means (e.g., text boxes, radio buttons, check boxes, etc.). Upon receiving the semen analysis results, the semen analysis component (108), if necessary, converts the semen analysis results into a format that is understood by the diagnostic matrix component (112). The semen analysis results (or in some cases the converted semen analysis results) are subsequently forwarded to the diagnostic matrix component (112) for analysis.

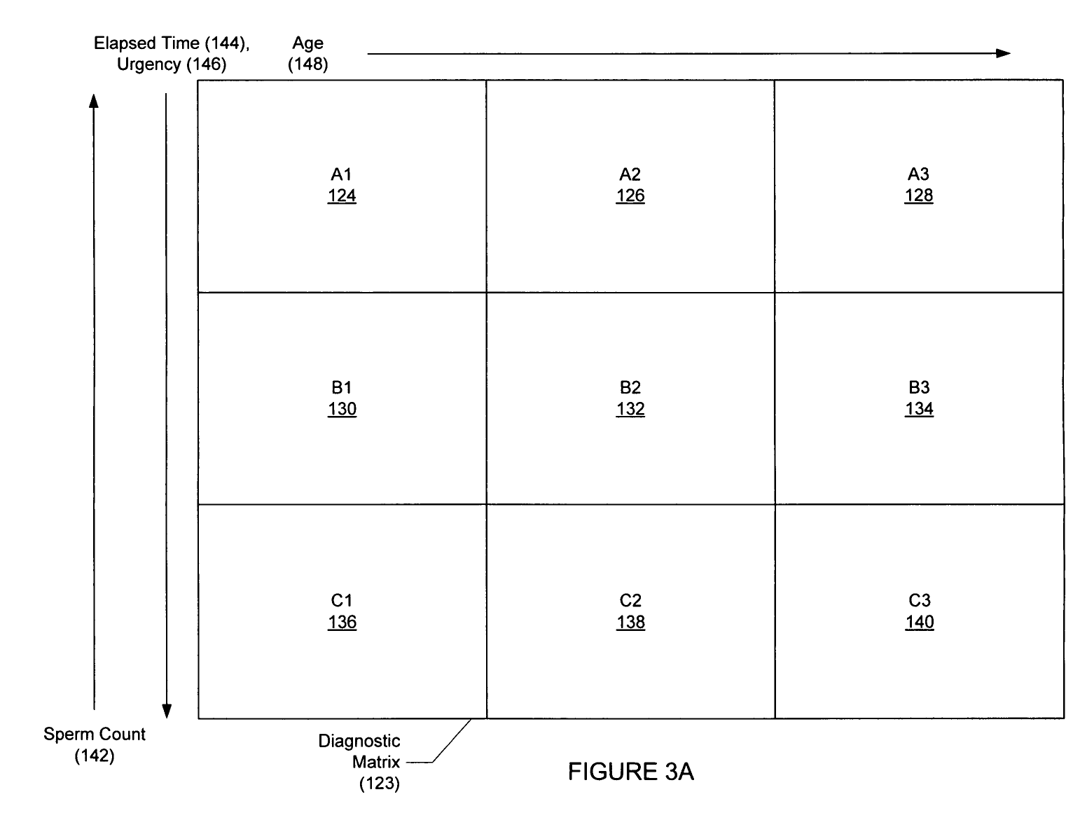

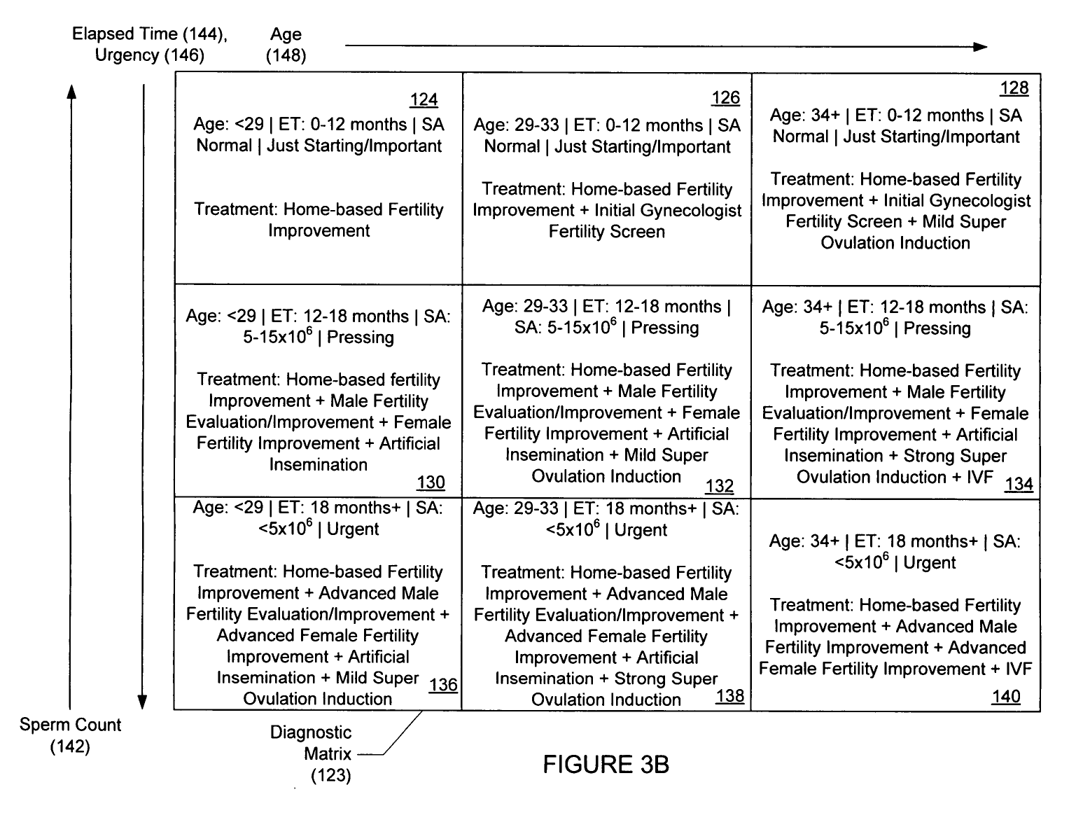

In one embodiment of the invention, the diagnostic matrix component (112) includes a diagnostic matrix (see FIGS. 3A-3B) and functionality to use the responses to the fertility survey and the semen analysis results to determine an initial location in the diagnostic matrix. In one embodiment of the invention, the diagnostic matrix component (112) analyzes the responses to the fertility survey to obtain a preliminary matrix location and then analyzes the semen analysis results to determine whether the preliminary matrix location should be changed to a more severe location in the diagnostic matrix (discussed in FIGS. 3A-3B). The result of the analysis of the responses to the fertility survey and the semen analysis results is the initial location in the diagnostic matrix.

Further, in one embodiment of the invention, the diagnostic matrix component (112) is configured to use information obtained from the information gathering components (110) in order to determine whether the location of the couple in the diagnostic matrix should be updated (discussed below).

In one embodiment of the invention, the diagnostic matrix component (112) also includes treatment options (discussed in FIGS. 3A-3B) associated with each of the locations in the diagnostic matrix. In addition, each location in the diagnostic matrix may include recommendations about specific combinations of treatment options as well as tests the male member and/or the female member should undergo in order to evaluate the source of the couple's infertility.

In one embodiment of the invention, the user may choose to pursue treatment options in addition to the ones associated with the location in the diagnostic matrix. In such cases, the user (via the client) is able to select additional treatment options. Further, in some cases, the user (via the client) may also choose not to pursue one or more of the treatment options associated with the location in the diagnostic matrix. In such cases, the user (via the client) is able to only select the treatment options he/she wishes to pursue.

Thus, the treatment options submitted to the ranking logic correspond to the treatment options associated with the location in the diagnostic matrix (and in some cases, only those treatment options selected by the user) as well as the additional selected treatment options. Once the treatment options are determined, the diagnostic matrix component (112) forwards the treatment options to the ranking logic (116).

In one embodiment of the invention, the ranking logic (116) is configured to rank the treatment options. In one embodiment of the invention, the ranking logic (116) uses the responses from the fertility survey, as well as information from the cost component (118), the probability component (120), and the timeframe component (122) to rank the various treatment options.

In one embodiment of the invention, the ranking logic (116) uses the responses of the fertility survey to determine: (i) the couple's urgency for children (i.e., how soon they want to have children), and (ii) the couple's sensitivity to cost, and (iii) the couple's willingness to try various medical procedures (e.g., will the couple consider IVF). The aforementioned information is used to adjust the values of the X, Y, and Z variables discussed below.

In one embodiment of the invention, the cost component (118) is configured to obtain the couple's insurance information (e.g., policy number, carrier, etc.). The couple's insurance information is subsequently used to determine the cost of the various treatment options (i.e., how much the insurance will pay for each treatment option and how much the couple must pay for each treatment option).

In one embodiment of the invention, the probability component (120) includes the probability of conceiving associated with each of the treatment options. In one embodiment of the invention, the probability of conceiving may be based on studies conducted by various medical research organizations or any other source. Further, the probabilities listed in the probability component (120) may be updated as additional statistical information is obtained.

In one embodiment of the invention, the time frame component (122) corresponds to the timeframe during which the treatment options increase the chance of conceiving. For example, for a particular treatment option, the timeframe to see an improvement in fertility and/or during which a treatment will be effective may be 3-6 months from the start of the treatment.

In one embodiment of the invention, the information from the aforementioned components along with the responses to the fertility survey are used by the ranking logic (116) to rank the various treatment options. In one embodiment of the invention, the ranking logic (116) performs the following steps to rank the various treatment options: (i) remove any treatment options that the couple is not willing to pursue based on the responses to the fertility survey; (ii) obtain the cost for each of the remaining treatment options using the cost component (118); (iii) obtain the probability of conception associated with each of the remaining treatment options from the probability component (120); (iv) obtain the timeframe associated with each of the remaining treatment options using the timeframe component (122); (v) calculate an aggregate value using the above information; and (vi) rank each of the treatment options using the aggregate values (e.g., ranking the treatment options from highest aggregate value to lowest aggregate value, where the highest aggregate value is the highest recommended treatment option).

In one embodiment of the invention, the aggregate value for a given treatment option is determined using the following equation:
aggregate value=[X*(cost of treatment option)]⁻¹*[Y*(probability of conception)]*[Z*(average timeframe)]⁻¹
where the cost of treatment option corresponds to the cost the couple will pay for the treatment option (taking into account the amount that their insurance will cover), the probability of conception corresponds to the probability of conception using the treatment option, the average timeframe corresponds to the average timeframe (e.g., 7.5 months if the timeframe is 6-9 months), and X, Y, and Z correspond to numbers (decimal or non-decimal) greater than or equal to zero.

In one embodiment of the invention, as discussed above, the values of the variables X, Y, Z, are adjusted depending on responses to the fertility survey. For example, if the couple does not care about the cost of the treatment option and is only concerned about the timeframe, then X would be set of a value of less than one and Y and Z would be set to equal values greater then X. The result of this adjustment would be that the treatment options with highest probability and shortest timeframes would be ranked higher than treatment options with longer timeframes and/or lower probabilities. Further, if two treatment options have the same probability and the same timeframe, then the treatment option with the lower cost would be ranked higher. Those skilled in the art will appreciate that the ranking logic (116) is not limited to the above equation. Those skilled in the art will also appreciate that the values of X, Y, and Z may be adjusted on a per user/couple basis using, for example, the user's responses to the fertility survey and the insurance information input by the user.

Once the ranked treatment options (114) have been determined by the ranking logic (116), the server (100) includes the necessary functionality to present the user (via the client) with the ranked treatment options. Once the user has selected one or more treatment options, then the user (via the client) may be provided with various progress tracking services supported by the various information gathering components (110) on the server (100).

In one embodiment of the invention, the information gathering components (110) are configured to track the progress of the couple as they pursue one or more treatment options. For example, the information gathering components (110) may include functionality to periodically request: (i) the female member to provide information about whether she has conceived, (ii) the female member to provide information about whether her ovulation has normalized and/or whether she has had a late period(s), (iii) the male member to provide information about semen analysis results obtained after the male member has been pursuing one or more treatment options for a particular period of time; (iv) the female and/or male member to provide test results for fertility related tests performed on the male member and/or female member; and (v) track the amount of time that has elapsed since the couple began pursuing one or more of the treatment options. Those skilled in the art will appreciate that the information gathering components (110) may also obtain/request information that is not listed above.

FIG. 3A shows a diagnostic matrix in accordance with one embodiment of the invention. More specifically, FIG. 3A shows an overview of the diagnostic matrix (123) in accordance with one embodiment of the invention. As shown in FIG. 3A, the diagnostic matrix (123) includes three rows (denoted A, B, and C) and three columns denoted (1, 2, 3). In one embodiment of the invention, each location in the matrix is designated by a row (e.g., A) and a column (e.g., 1). Accordingly, there are nine locations in the diagnostic matrix (123): A1 (124), A2 (126), A3 (128), B1 (130), B1 (132), B2 (134), B3 (136), C1 (138), C2, (140), and C3 (142) (described below in FIG. 3B). In one embodiment of the invention, the level of severity of infertility increases as one proceeds down the rows and across the columns. Thus, the location designated as A1 (124) is the least severe and the location designated as C3 (140) is the most severe.

As shown in FIG. 3A, the diagnostic matrix (123) is associated with the following four fertility factors: age (i.e., age of the female member) (148), elapsed time (i.e., amount of time the couple has been trying to conceive) (144), urgency (i.e., how urgent is the couple's desire to conceive) (146), and sperm count (e.g., total motile sperm count) (142). In one embodiment of the invention, each location in the diagnostic matrix (123) corresponds to a unique combination of values for each of the aforementioned fertility factors and is associated with one or more treatment options.

Those skilled in the art will appreciate that while the diagnostic matrix (123) has been described as having nine locations and correlated with four fertility factors, the invention may be implemented using a larger or smaller diagnostic matrix which correlates a greater number or a smaller number of fertility factors.

FIG. 3B shows a diagnostic matrix in accordance with one embodiment of the invention. More specifically, FIG. 3B shows the unique combination fertility factor values associated with each of the locations in the diagnostic matrix (123) as well as the proposed treatment options.

As shown in FIG. 3B, location A1 (124) is associated with the following four fertility factor values: (i) Age: <29; (ii) Elapsed Time (ET): 0-12 months; (iii) Semen Analysis Results (SA): Normal (e.g., TMC>15*10⁶ and no presence of bacteria, white blood cells, red blood cells or agglutination in semen); and (iv) Urgency: just start/important.

In one embodiment of the invention, the treatment option associated with the location A1 (124) is home-based fertility improvement. In one embodiment of the invention, home-based fertility improvement includes (i) monitoring ovulation using at least one of the following methods: basal body temperature, cervical mucus testing, and an ovulation predictor kit; (ii) timed intercourse; (iii) lifestyle changes for the male member and the female member of the couple (e.g., substances to avoid, increased exercise, etc.); and (iv) charts for tracking improvement in fertility. Those skilled in the art will appreciate that the home-based fertility improvement may include additional components or fewer components than the ones listed above.

Continuing with the discussion of FIG. 3B, location A2 (126) is associated with the following four fertility factor values: (i) Age: 29-30; (ii) Elapsed Time (ET): 0-12 months; (iii) Semen Analysis Results (SA): Normal (e.g., TMC>15*10⁶); and (iv) Urgency: just start/important.

In one embodiment of the invention, A2 (126) is associated with two treatment options: (i) home-based fertility improvement (discussed above) and (ii) an initial gynecologist fertility screen. In one embodiment of the invention, the initial gynecologist fertility screen may include one or more of the following: (i) advanced ovulation monitoring (e.g., ovulation monitoring by the gynecologist using, for example, blood tests and ultra sounds); (ii) determination of fallopian tube quality (e.g., an evaluation of fallopian tube openness); and (iii) an evaluation to determine the presence of uterine abnormalities (e.g., an evaluation to determine the presence of uterine scar tissue, fibroids, developmental abnormalities (e.g., double uterus), etc.). Those skilled in the art will appreciate that the above list is not intended to limit the scope of the initial gynecologist fertility screen.

Continuing with the discussion of FIG. 3B, location A3 (128) is associated with the following four fertility factor values: (i) Age: >34; (ii) Elapsed Time (ET): 0-12 months; (iii) Semen Analysis Results (SA): Normal (e.g., TMC>15*10⁶); and (iv) Urgency: just start/important.

In one embodiment of the invention, A3 (128) is associated with the following treatment options: (i) home-based fertility improvement (discussed above), (ii) an initial gynecologist fertility screen (described above), and (iii) mild super ovulation induction. In one embodiment of the invention, mild super ovulation induction corresponds to using oral ovulation medications such as Clomid® (Clomid is a registered trademark of Merrell Dow Pharmaceuticals Inc.) to increase the number of eggs released during each menstrual cycle.

Continuing with the discussion of FIG. 3B, location B1 (130) is associated with the following four fertility factor values: (i) Age: <29; (ii) Elapsed Time (ET): 12-18 months; (iii) Semen Analysis Results (SA): TMC 5-15*10⁶; and (iv) Urgency: pressing.

In one embodiment of the invention, A3 (128) is associated with the following treatment options: (i) home-based fertility improvement (discussed above); (ii) male fertility evaluation/improvement; (iii) female fertility improvement; and (iv) artificial insemination (A1). In one embodiment, male fertility evaluation/improvement may correspond to one or more of the following: (i) having a urologist examine the male member's reproductive organs (e.g., penis, urethra, prostate, spermatic cord, testicles, and tubes that drain the testicles for any factors that may contribute to infertility) and (ii) taking medications and/or having medical procedures to address infertility factors discovered by the urologist. Those skilled in the art will appreciate that the above list is not intended to limit the scope of male fertility evaluation/improvement.

In one embodiment of the invention, female fertility improvement corresponds to an initial gynecologist fertility screen (if one has not been conducted) as well as continuing to consult with a gynecologist in an attempt to improve female fertility. The continued consultation with the gynecologist may correspond to a review of the female member's menstrual history, evaluation of the female member's cervix, uterus, fallopian tubes, ovaries, and peritoneum using x-ray studies and ultrasound, etc. In addition, the gynecologist may also initiate treatments (medicinal, surgical, or a combination thereof) to improve ovulation, structural integrity and/or function of the female reproductive tract. In one embodiment of the invention, A1 corresponds to injecting the male member's sperm in the female member's uterus. A1 is a medical procedure and, thus, is overseen by a medical professional (e.g., a gynecologist) trained to perform the procedure.

Continuing with the discussion of FIG. 3B, location B2 (132) is associated with the following four fertility factor values: (i) Age: 29-33; (ii) Elapsed Time (ET): 12-18 months; (iii) Semen Analysis Results (SA): TMC 5-15*10⁶; and (iv) Urgency: pressing.

In one embodiment of the invention, B2 (132) is associated with the following treatment options: (i) home-based fertility improvement (discussed above); (ii) male fertility evaluation/improvement (discussed above); (iii) female fertility improvement (discussed above); (iv) artificial insemination (A1) (discussed above); and (v) mild super ovulation induction (discussed above).

Continuing with the discussion of FIG. 3B, location B3 (134) is associated with the following four fertility factor values: (i) Age: >34; (ii) Elapsed Time (ET): 12-18 months; (iii) Semen Analysis Results (SA): TMC 5-15*10⁶; and (iv) Urgency: pressing.

In one embodiment of the invention, B3 (134) is associated with the following treatment options: (i) home-based fertility improvement (discussed above); (ii) male fertility evaluation/improvement (discussed above); (iii) female fertility improvement (discussed above); (iv) artificial insemination (A1) (discussed above); (v) strong super ovulation induction; and (vi) IVF.

In one embodiment of the invention, strong super ovulation induction corresponds to using injectable ovulation drugs that include, for example, gonadatropins, to increase the number of eggs released during each menstrual cycle. In one embodiment of the invention, IVF corresponds to fertilizing an egg from the female member of the couple with sperm from the male member of the couple. The fertilized egg is then implanted into the uterus of the female member. IVF is a medical procedure and, thus, is overseen by a medical professional (e.g., an IVF specialist) trained to perform the procedure.

Continuing with the discussion of FIG. 3B, location C1 (136) is associated with the following four fertility factor values: (i) Age: <29; (ii) Elapsed Time (ET): 18+ months; (iii) Semen Analysis Results (SA): TMC<5*10⁶; and (iv) Urgency: urgent.

In one embodiment of the invention, C1 (136) is associated with the following treatment options: (i) home-based fertility improvement (discussed above); (ii) advanced male fertility evaluation/improvement; (iii) advanced female fertility improvement; (iv) artificial insemination (AI) (discussed above); and (v) mild super ovulation induction.

In one embodiment of the invention, the advanced male fertility evaluation/improvement includes the male fertility evaluation/improvement discussed above as well as a reevaluation of the male member by a reproductive urologist. In one embodiment of the invention, the reproductive urologist performs a complete genetic evaluation for the male member of the couple. After the re-evaluation, the male member may be instructed to take additional actions and/or medications by the reproductive urologist in order to increase TMC of the male member's semen.

In one embodiment of the invention, advanced female fertility improvement includes the female fertility improvement discussed above as well as additional procedures and/or actions that are undertaken to establish regular ovulation, open fallopian tubes, remove fibroids, and/or treat severe endometriosis. Note that at this stage, treatment options associated with advanced female fertility improvement includes treatment for severe versions of female reproductive diseases.

Continuing with the discussion of FIG. 3B, location C2 (138) is associated with the following four fertility factor values: (i) Age: 29-33; (ii) Elapsed Time (ET): 18+ months; (iii) Semen Analysis Results (SA): TMC<5*10⁶; and (iv) Urgency: urgent.

In one embodiment of the invention, C2 (138) is associated with the following treatment options: (i) home-based fertility improvement (discussed above); (ii) advanced male fertility evaluation/improvement (discussed above); (iii) advanced female fertility improvement (discussed above); (iv) artificial insemination (A1) (discussed above); and (v) strong super ovulation induction (discussed above).

Continuing with the discussion of FIG. 3B, location C3 (140) is associated with the following four fertility factor values: (i) Age: 34+; (ii) Elapsed Time (ET): 18+ months; (iii) Semen Analysis Results (SA): TMC<5*10⁶; and (iv) Urgency: urgent.

In one embodiment of the invention, C3 (140) is associated with the following treatment options: (i) home-based fertility improvement (discussed above); (ii) advanced male fertility evaluation/improvement (discussed above); (iii) advanced female fertility improvement (discussed above); and (iv) IVF (discussed above).

FIG. 4A shows a flowchart in accordance with one embodiment of the invention. More specifically, the flowchart in FIG. 4A details the questions asked in a fertility survey in accordance with one embodiment of the invention. Further, as shown in FIG. 4A, depending on the user's responses (i.e., responses of the male and/or female member of the couple) to the fertility survey, the user (which in some cases is the couple) is associated with a particular location (e.g., A1) or a particular row (e.g., B) in the diagnostic matrix. As the user proceeds through the survey (i.e., answers the questions in the fertility survey) the user is associated with a specific location or a row in the diagnostic matrix. Once the user is associated with a particular location (or row) in the diagnostic matrix, the user's location (or row) is not changed with a subsequent response to a question, unless the response moves the user to a more severe location (e.g., from B1 to B2 or to a more severe row (e.g., from A to B).

As discussed above, the analysis of the particular responses is performed by the matrix diagnostic component; accordingly, the flowchart shown in FIG. 4A includes the questions stored in the fertility survey component as well as the analysis logic included in the diagnostic matrix component. Further, as discussed above, the result of the analysis of the responses to the fertility survey corresponds to a preliminary location in the diagnostic matrix. In one embodiment of the invention, information about three fertility factors, namely, female member's age, urgency, and elapsed time, along with the female member's medical history are used to determine the preliminary location. Thus, if the female has a normal medical history (i.e., no medical conditions and/or history that would impact her ability to conceive), then the preliminary location is determined using only the female member's age, urgency, and elapsed time.

Turning to FIG. 4A, initially, the fertility survey requests information about the female member's body mass index (BMI) (ST100). In one embodiment of the invention, the BMI is determined by obtaining the female member's weight and height. The user is then asked to provide the female member's age (ST102), the urgency (e.g., important, pressing, urgent) of the couple to conceive (ST104), and the financial concerns (e.g., budget conscious, balanced, result oriented (i.e., cost is not important)) of the couple (ST106).

The user is subsequently asked to provide information about the willingness of the couple to try various medical procedures (e.g., willingness to try IVF) (ST108), the number of months the couple has been trying to conceive (ST110), whether the female member has previously had a normal delivery with birth defects (ST112), and whether the female member has ever had any stillbirths (ST114). At this stage, the responses to the fertility survey may be used to place the user in a row within the diagnostic matrix, a specific location, and also indicate which specific locations in the diagnostic matrix (e.g., C3) the user cannot be placed.

The following questions request additional information from the user in order to fine tune the placement of the user within the diagnostic matrix: has the female member previously had two or more medically documented miscarriages in the last five years? (ST116) (Note: “drop one row” corresponds to moving the couple from A to B or from B to C. If the couple is already at C, then they just remain there); has the female member previously had an ectopic pregnancy? (ST118); is the female member experiencing regular menstrual cycles? (ST120); and is the female member experiencing painful menstrual cycles? (ST122).

In addition to the above questions, the user is also requested to provide information related to the medical history of the male member and female member of the couple by providing the following information: male fertility tests received (ST124) (see FIG. 4B); male fertility diagnoses received (ST126) (see FIG. 4C); female fertility diagnoses received (ST128) (see FIG. 4D); female treatment received (ST130) (see FIG. 4E); and additional female medical history (ST132) (see FIG. 4F). In one embodiment of the invention, the information obtained in ST124 and ST126 is not used in the determination of a preliminary location in the matrix; rather, the information obtained in ST124 and ST126 is used to adjust the initial location in the matrix (discussed below) and/or in the development of a fertility treatment plan.

At this stage the preliminary location in the diagnostic matrix is known. As discussed above, once the preliminary location in the diagnostic matrix is obtained, the diagnostic matrix component uses the results of semen analysis to determine a row (e.g., A, B, or C) in the diagnostic matrix. In one embodiment of the invention, the semen analysis is used to determine the total motile sperm count (TMC) (i.e., total sperm count*% of motile sperm).

For example, if the aforementioned row in the diagnostic matrix is lower than or equal to the row of the preliminary location, then the preliminary location corresponds to the initial location. For example, if the preliminary location is B2 and the row determined using the semen analysis is A, then the initial location is B2. However, if the row is greater than the row of the preliminary location, then the preliminary location is updated to a more severe location. For example, if the preliminary location is B2 and the row determined using the semen analysis is C, then the initial location is C2.

In one embodiment of the invention, if the sperm morphology is poor (e.g., the shape of the sperm is poor (as defined by accepted medical standards) or there is bacteria, white blood cells, red blood cells and/or agglutination is present), then the initial location may be adjusted.

In one embodiment of the invention, if the morphology of the sperm is poor (discussed above), then the aforementioned preliminary location may be adjusted to a more severe location in the diagnostic matrix. For example, if the preliminary location is B2 and the results of the semen analysis correspond to B, then the initial location is B2. However, if the sperm morphology is poor, then the initial location is set to B3.

As an alternative to the aforementioned semen analysis methodology (i.e., using TMC and then adjusting based on morphology and the presence of bacteria, white blood cells, and/or red blood cells), the following semen analysis methodology may be used.

Initially, six categories are established: (i) super fertile (TMC≧50 million sperm); (ii) moderate fertility (TMC=approx. 20-50 million sperm); (iii) low fertility (TMC=approx. 12-20 million sperm); mild subnormal fertility (TMC=approx. 7-12 million sperm); (iv) moderate subnormal fertility (TMC approx. 5-7 million sperm); and (vi) severe subnormal fertility (TMC approx. <5 million sperm). Based on the semen analysis, the male member is placed within one of the aforementioned categories. Those skilled in the art will appreciate that the aforementioned categories may be associated with different TMC ranges.

Once the male member is placed in one of the six categories, the morphology is used to determine whether to move the male member from the current category to a more severe category. More specifically, if morphology (i.e., percentage of sperm with a normal shape (as defined by accepted medical standards)) is greater than 30%, then the male member is not moved from the current category. However, if the morphology is less than or equal to 30%, then the male member is moved to a more severe category based on the following table:

Morphology Adjustment

| Morphology | Adjustment to Category |
|---|---|
| 20-30% | Move down up to one category |
| 15-20% | Move down up to two categories |
| 10-15% | Move down up to three categories |
| 5-10% | Move down up to four categories |
| <5% | Move down up to five categories |

Once the category adjustments have been made based on the morphology, the male member is associated with a row (i.e., A, B, or C) in the diagnostic matrix based on the following mapping between category and rows in the diagnostic matrix.

Category to Row Mapping

| Category | Row |
|---|---|
| Super Fertile | A |
| Moderate Fertility | A |
| Low Fertility | B |
| Mild Subnormal Fertility | B |
| Moderate Subnormal Fertility | C |
| Severe Subnormal Fertility | C |

Once the initial row is determined using the above mapping, the row may be adjusted based on the following additional factors: (i) if in A and there is the presence of bacteria, white blood cells, and/or red blood cells, then move from row A to B; (ii) if there is agglutination (i.e., as a result of the presence of antisperm antibodies), then move from current row to row C (unless already in row C); and (iii) if there is low volume (e.g., <2.0 cc) in the sample provided from semen analysis, then move to row B (unless already in row B or row C).

In one embodiment of the invention, the male medical history obtained in the fertility survey (see ST124, ST126, and FIGS. 4B and 4C) also may be used to adjust the initial location in the matrix. For example, if the initial location in the matrix is A2 (determined by using the fertility survey, semen analysis, and morphology) but the male member has a hormone imbalance, then the initial location may be adjusted to B1.

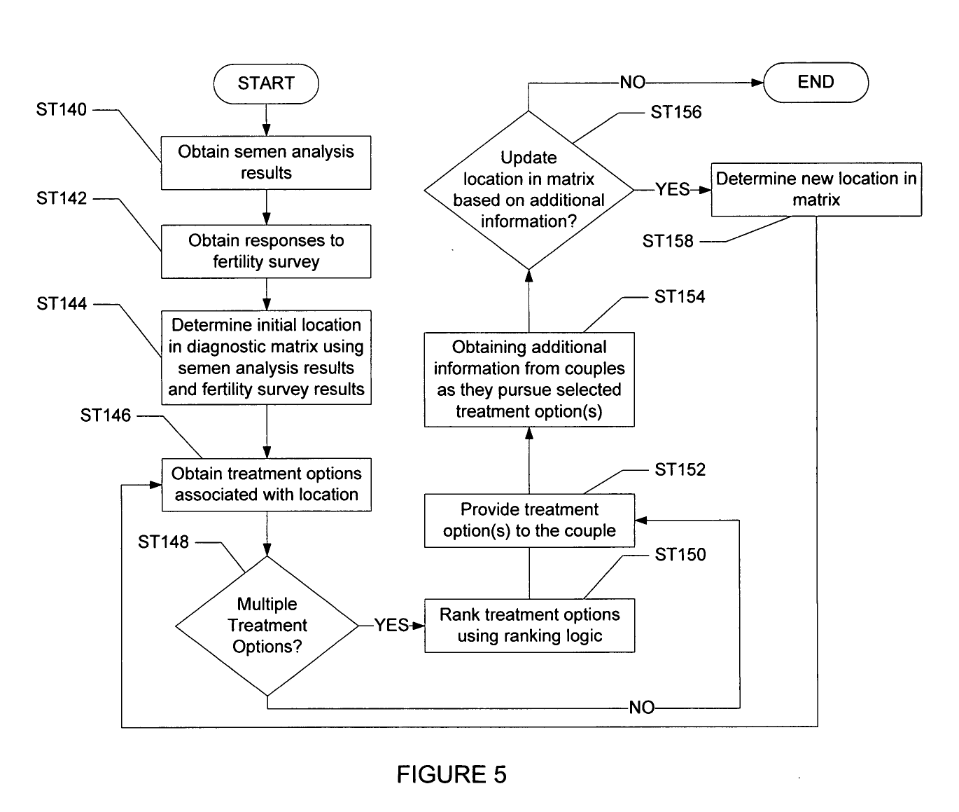

FIG. 5 shows a flowchart in accordance with one embodiment of the invention. Initially, semen analysis results (ST140) and responses to the fertility survey (ST142) are obtained. The semen analysis results and the fertility survey results are then used to determine (as discussed above) an initial location in the diagnostic matrix for the couple (ST144).

Once the initial location has been determined, the treatment options associated with the initial location are obtained (ST144). Though not shown in FIG. 4, ST144 may also include allowing the user to select additional treatment options or elect not to purse certain treatment options (as discussed above). Once the treatment options have been determined, if multiple treatment options are obtained in ST144 (ST148), then treatment options are ranked using the ranking logic (ST150). The ranked treatment options are then provided/presented, for example via the client, to the couple (ST152). Alternatively, if only one treatment option is provided in ST144 or if the user does not wish to rank the treatment options, then ST150 is not performed, and the treatment options, without any ranking, are presented to the couple (ST150).

At this stage, the couple then proceeds to follow one or more of the treatment options presented in ST150 and provide information related to fertility as they pursue one or more of the treatment options (ST154). Depending on the information obtained in ST154 (ST156), the couple may be moved from their current location in the diagnostic matrix to a less severe or more severe location in the diagnostic matrix (ST158). If the couple is moved to a new location in the diagnostic matrix, then the flowchart proceeds to ST146. Alternatively, if the couple's location in the matrix does not need to be updated, then the flowchart ends.

In one embodiment of the invention, the couple may be moved to a new location in the diagnostic matrix if (i) a certain amount of time has elapsed since the treatment option has been pursed and the couple has not conceived and (ii) the results of a specific test indicate that the couple should be placed in a new matrix location (e.g., the semen analysis results have not improved with the current treatment option, the female member blood test reveals that the FSH/E2 levels are abnormal, if an examination for uterine abnormalities reveals that the female member has severe uterine abnormalities, etc.).

In one embodiment of the invention, the couple moves to a more severe row (e.g., from row A to B) after 6-12 months of pursuing the treatment options and not successfully conceiving. Note that the timeframe to move from one row to another may vary between rows. In one embodiment of the invention, the couple moves to a more severe location in a row (e.g., A1 to A2) if the results of the tests performed as part of the treatment option in the given location show that the couple should be in a more severe location in the row. For example, if tests reveal that the female member's ovulation is not normalizing after pursuing certain treatment options associated with the AI, then the couple may move to A2.

Depending on the information gathered by the information gathering components, the couple may be moved to a more severe location in the same column within the diagnostic matrix (e.g., from A1 to B1), to a more severe location in the same row within the diagnostic matrix (e.g., from A1 to A2), or to a more severe location in a different row and a different column within the diagnostic matrix (e.g., from A1 to B2). Those skilled in the art will appreciate that, depending on the information gathered, the couple may be moved from their current location in the diagnostic matrix to any location that is more severe in the diagnostic matrix.

One or more embodiments of the invention provide a balanced approach to assessing and treating infertility. Specifically, using the methods and systems described above, both of the members of the couple (i.e., the male member and the female member) are diagnosed and treated in an effort to increase the couple's overall fertility and probability of conceiving.

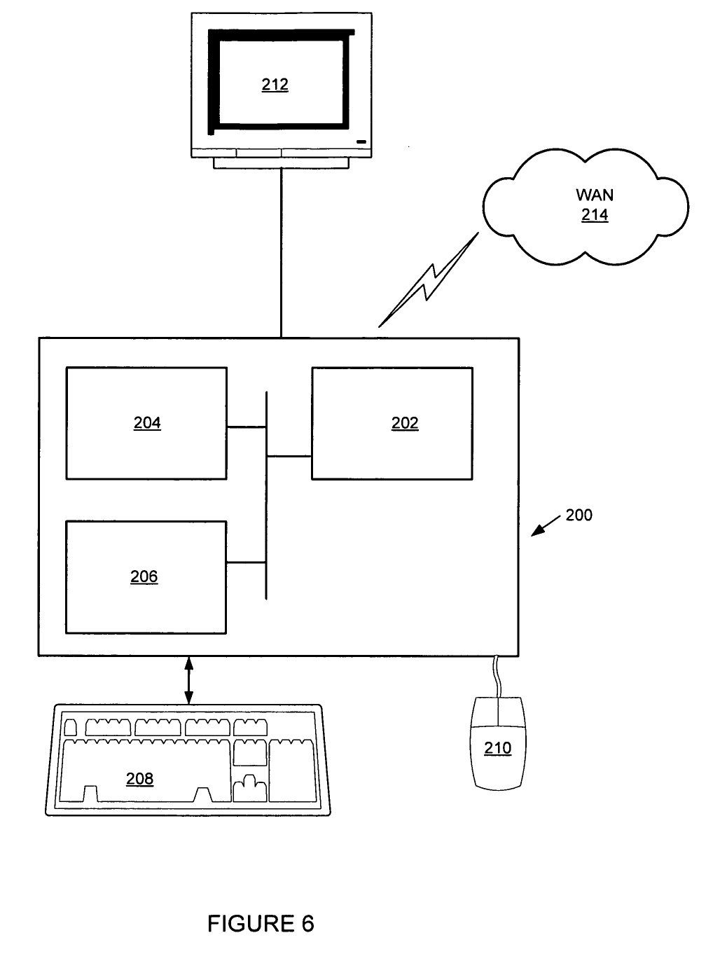

The invention may be implemented on virtually any type of computer regardless of the platform being used. For example, as shown in FIG. 6, a networked computer system (200) includes a processor (202), associated memory (204), a storage device (206), and numerous other elements and functionalities typical of today's computers (not shown). The networked computer (200) may also include input means, such as a keyboard (208) and a mouse (210), and output means, such as a monitor (212). The networked computer system (200) is connected to a local area network (LAN) or a wide area network (214) (e.g., the Internet) via a network interface connection (not shown). Those skilled in the art will appreciate that these input and output means may take other forms. Those skilled in the art will appreciate that one or more elements of the aforementioned computer (200) may be located at a remote location and connected to the other elements over a network. Further, software instructions to perform embodiments of the invention may be stored on a computer readable medium such as a compact disc (CD), a diskette, a tape, a file, or any other computer readable storage device.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method for assessing fertility for a couple comprising:
obtaining semen analysis results from a male member of the couple;
obtaining fertility survey responses from at least a female member of the couple;
determining an initial location in a diagnostic matrix using the semen analysis results and the fertility survey responses; and
obtaining a plurality of treatment options corresponding to the initial location in the diagnostic matrix.

2. The method of claim 1, further comprising:
tracking progress of the couple as the couple pursues at least one of the plurality of treatment options.

3. The method of claim 2, wherein tracking the progress of the couple comprises at least one selected from the group consisting of obtaining results of a subsequent semen analysis after the at least one treatment option has been pursued for a specified duration, obtaining a result of a test performed on the female member, determining whether the couple conceived, and an determining amount of time elapsed since the couple began pursuing the at least one treatment option.

4. The method of claim 2, further comprising:
moving the couple from the initial location in the matrix to a new location in the matrix based on the progress of the couple.

5. The method of claim 1, further comprising:
ranking the plurality of treatment options.

6. The method of claim 5, further comprising:
removing at least one of the plurality of treatment options prior to ranking the plurality of treatment options, if the fertility survey results indicate that the couple will not pursue the at least one of the plurality of treatment options.

7. The method of claim 5, wherein the treatment options are ranked based on the cost associated with each of the plurality of treatment options, the probability of conception associated with each of the plurality of treatment options, and the timeframe for conception associated with each of the plurality of treatment options.

8. The method of claim 7, wherein the cost associated with each of the plurality of treatment options is determined using the couple's insurance information.

9. The method of claim 1, wherein the diagnostic matrix comprises a plurality of locations, wherein the initial location is one of the plurality of locations, wherein each of the plurality of locations is associated with a unique combination of fertility factor values.

10. The method of claim 9, wherein the fertility factor values comprise a female member's age value, an urgency value, a sperm count value, and an elapsed time value.

11. The method of claim 10, wherein the sperm count value corresponds to a total motile sperm count.

12. The method of claim 1, wherein determining the initial location in the diagnostic matrix comprises:
determining a preliminary location in the diagnostic matrix using the fertility survey analysis results;
if the preliminary location is more severe than a matrix location determined using the semen analysis results only, then the preliminary location is the initial location; and
if the preliminary location is less severe than a matrix location determined using only the semen analysis results, then the matrix location determined using only the semen analysis results is the initial location.

13. The method of claim 12, wherein the matrix location determined using only the semen analysis results is the matrix location determined using only a total motile sperm count if the sperm morphology is normal, and is the matrix location determined using the total motile sperm count with an adjustment taking into account the sperm morphology if the sperm morphology is abnormal.

14. A system for assessing fertility for a couple comprising:
a fertility survey component configured to obtain responses of a fertility survey from at least a female member of the couple;
a semen analysis component configured to obtain results of semen analysis from a male member of the couple; and
a diagnostic matrix component comprising a diagnostic matrix and configured to:
determine an initial location in a diagnostic matrix using the semen analysis results and the fertility survey responses, and
obtain a plurality of treatment options corresponding to the initial location in the diagnostic matrix.

15. The system of claim 14, further comprising:
an information gathering component configured to track progress of the couple as the couple pursues at least one of the plurality of treatment options.

16. The system of claim 15, wherein tracking the progress of the couple comprises at least one selected from the group consisting of obtaining results of a subsequent semen analysis after the at least one treatment option has been pursued for a specified duration, obtaining a result of a test performed on the female member, determining whether the couple conceived, and determining an amount of time elapsed since the couple began pursuing the at least one treatment option.

17. The system of claim 15, wherein the diagnostic matrix component is further configured to move the couple from the initial location in the matrix to a new location in the matrix based on the progress of the couple.

18. The system of claim 14, further comprising:
ranking logic configured to rank the plurality of treatment options.

19. The system of claim 18, wherein the ranking logic is configured to remove at least one of the plurality of treatment options prior to ranking the plurality of treatment options, if the fertility survey responses indicate that the couple will not pursue the at least one of the plurality of treatment options.

20. The system of claim 18, wherein the treatment options are ranked based on the cost associated with each of the plurality of treatment options, the probability of conception associated with each of the plurality of treatment options, and the timeframe for conception associated with each of the plurality of treatment options.

21. The system of claim 20, wherein the cost associated with each of the plurality of treatment options is determined using the couple's insurance information.

22. The system of claim 14, wherein the diagnostic matrix comprises a plurality of locations, wherein the initial location is one of the plurality of locations, wherein each of the plurality of locations is associated with a unique combination of fertility factor values.

23. The system of claim 22, wherein the fertility factor values comprise a female member's age value, an urgency value, a sperm count value, and an elapsed time value.

24. The system of claim 23, wherein the sperm count value corresponds to a total motile sperm count.

25. The system of claim 14, wherein determining the initial location in the diagnostic matrix comprises:
determining a preliminary location in the diagnostic matrix using the fertility survey analysis results;
if the preliminary location is more severe than a matrix location determined using the semen analysis results then the preliminary location is the initial location; and
if the preliminary location is less severe, than a matrix location determined using only the semen analysis results, then the matrix location determined using only the semen analysis results is the initial location.

26. The system of claim 25, wherein the matrix location determined using only the semen analysis results is the matrix location determined using only a total motile sperm count if the sperm morphology is normal, and is the matrix location determined using the total motile sperm count with an adjustment taking into account the sperm morphology if the sperm morphology is abnormal.

27. A computer readable medium comprising instructions to:
obtain semen analysis results from a male member of the couple;
obtain fertility survey responses from at least a female member of the couple;
determine an initial location in a diagnostic matrix using the semen analysis results and the fertility survey responses;
obtain a plurality of treatment options corresponding to the initial location in the diagnostic matrix;
rank the plurality of treatment options;
track progress of the couple as the couple pursues at least one of the plurality of treatment options; and
move the couple from the initial location in the matrix to a new location in the matrix based on the progress of the couple.
